(19) United States
(12) Patent Application Publication
(10) Pub. No.: US 2019/0090828 A1
(43) Pub. Date: Mar. 28, 2019

(54) X-RAY EXAMINATION DEVICE AND METHOD

(71) Applicant: SIEMENS HEALTHCARE GMBH, Erlangen (DE)

(72) Inventors: DAVID DEDERICHS, POXDORF (DE); RICCARDO KUNZE, ERLANGEN (DE); CHRISTOPH LOETZSCH, BAMMERSDORF (DE); MARCUS RADICKE, VEITSBRONN (DE)

(21) Appl. No.: 16/142,191

(22) Filed: Sep. 26, 2018

(30) Foreign Application Priority Data
Sep. 26, 2017 (EP) 17193206.4

(51) Int. Cl. A61B 6/00; A61B 6/04; A61B 8/00; A61B 8/08

(52) U.S. Cl. CPC A61B 6/4417; A61B 6/0414; A61B 6/502; A61B 8/403; A61B 6/463; A61B 6/0485; A61B 8/4416; A61B 8/463; A61B 8/0825

(57) ABSTRACT

An X-ray examination device has an X-ray tube and an X-ray detector for recording at least one X-ray image of a breast and also an ultrasound system for recording an ultrasound image of the breast. A compression unit is used for compression and fixation of the breast. A volume-variable and/or shape-variable positioning element is disposed to act on the breast. The positioning element has at least one chamber and the volume-variability and/or shape-variability of the positioning element can be effected by filling at least one chamber of the positioning element with a filling medium and/or by emptying at least one chamber of the positioning element.

### CROSS-REFERENCE TO RELATED APPLICATION

This application claims the priority, under 35 U.S.C. § 119, of European patent application EP 17193206.4, filed Sep. 26, 2017; the prior application is herewith incorporated by reference in its entirety.

### BACKGROUND OF THE INVENTION

### Field of the Invention

The invention relates to an X-ray examination device and a method for positioning a breast in an X-ray examination device.

Early detection of breast cancer is a huge challenge for all currently existing medical imaging methods.

X-ray mammography and X-ray tomosynthesis examinations are two standard methods in breast cancer diagnostics. In both procedures, the patient is usually positioned in a standing position and the breast compressed in two different directions (cranio-caudal and medio-lateral or medio-lateral-oblique). Compression of the breast is above all of particular importance for mammography since it reduces the thickness of the overlying breast tissue and thus enables improved diagnostics to be achieved.

Herein, compression of the breast is typically carried out in such a way that the breast is positioned on the X-ray detector and a compression plate is pressed onto the breast from above with a force of up to 200 N. A major disadvantage of this type of compression is the pain that is often experienced by patients.

Therefore, a first aspect of the present invention relates to the reduction or avoidance of pain during compression or fixation of the breast during X-ray examinations.

There is widespread consensus through the field of medical research that diagnosis with simultaneously high degrees of sensitivity and specificity can only be obtained with a skillful combination of different imaging methods. Therefore, in addition to X-ray images, diagnosis by means of imaging methods frequently also employs ultrasound images.

As a rule, to this end, first an X-ray examination, for example tomosynthesis, is performed. In tomosynthesis, 2D X-ray images of a breast are recorded at different recording angles, for example during a circular-arc trajectory of the X-ray source around the object and reconstructed into a 3D dataset. This 3D dataset is then used as the basis for the creation of slice images or X-ray images with any technique for slicing the 3D dataset.

Then, optionally ultrasound images are additionally prepared to enable better evaluation of a tissue change for diagnostic purposes.

During an X-ray mammography examination, the patient usually stands or sits directly in front of the X-ray examination device and the breast is compressed between two plates as described above, while, during an ultrasound examination (sonography), the patient lies on a patient bench and the breast is pressed by the ultrasound transducer onto the thoracic cage. This means the patient has to be repositioned between the X-ray examination and the ultrasound examination. An additional disadvantage results from the fact that the breast is compressed to different degrees and in different directions during the X-ray examination and the ultrasound examination. A further disadvantage is the fact that the X-ray images and the ultrasound images are recorded from different viewing directions and it is therefore difficult to superimpose or combine the images obtained in this way as precisely as possible.

Prototype combined X-ray/ultrasound examination devices are known. For example, examination devices are known with which a compression arrangement includes a lower compression unit in the form of a bearing plate comprising the X-ray detector and an upper compression unit in the form of a compression plate, for example a plexiglass plate. Such devices are used for an ultrasound examination on a breast that is compressed between the upper compression plate and the lower bearing plate of the examination device. Herein, the ultrasound transducer required for sonography is guided in a scanning movement along the tomosynthesis scan direction over the upper side of the compression plate. Due to the substantially unchanged compression of the breast during both of the imaging methods and due to the defined, and hence correlatable, viewing directions, the images obtained enable improved diagnosis.

Since the radiated ultrasound signals have to be coupled through the compression plate into the breast to be examined, it has been suggested that a compression trough with a flexible compression surface be used instead of a rigid compression plate, wherein this flexible compression surface is, for example, made of a fabric known as gauze. In this embodiment of the compression trough, the breast adopts a slightly convex shape during compression. One advantage of this type of the compression is the more homogeneous distribution of the compressive force on the object to be examined and the possibility of performing an ultrasound examination immediately following an X-ray examination without the technical problems that would result from coupling of the ultrasound through a compression plate.

The major problem with hybrid systems that accomplish both an X-ray and an ultrasound examination in one single position of the breast relates to the coupling of the breast to the ultrasound transducer and hence to achieving good coverage of the breast surface during the ultrasound recording. Typically, despite compression, regions of the breast on which the compression plates do not act on both sides, i.e. in particular the region close to the breast wall near to the nipple, or, in other words, the anterior side of the breast, are not coupled to the ultrasound transducer at all or only unsatisfactorily coupled thereto so that these regions cannot be depicted by imaging. While, during the X-ray imaging, it is possible to record images of the entire breast arranged on the X-ray detector, with the corresponding ultrasound imaging with identical positioning of the breast, the anterior region of the breast, which can amount to up to 25 percent of the corresponding image area of the X-ray imaging, is omitted. As a result, the hybrid systems known to date can often only be used to a limited extent for screening or diagnostic purposes.

Therefore, a second aspect of the present invention relates to improved ultrasonic coupling with a combined X-ray/ultrasound examination.

German published patent application DE 199 01 724 A1 discloses a coupling unit for breast examinations in an X-ray mammography device with simultaneous breast diagnosis by means of ultrasound, wherein coupling cushions that can be filled with water via pumps are attached to the compression plate and/or bearing plate and, due to lateral supports, undergo deformation during filling so that they enclose the breast on all sides. Herein, compression of the breast is exclusively induced by the plates.

### SUMMARY OF THE INVENTION

It is accordingly an object of the invention to provide an X-ray examination device and a method which overcome the above-mentioned and other disadvantages of the heretofore-known devices and methods of this general type and that optimizes X-ray examinations of the breast with a view to at least the aspects of the invention mentioned above.

With the foregoing and other objects in view there is provided, in accordance with the invention, an X-ray examination device being a combined X-ray and ultrasound examination device, the X-ray examination device comprising:

an X-ray tube and an X-ray detector for recording an X-ray image of a breast;

an ultrasound unit for recording an ultrasound image of the breast; and

a compression unit for compression and fixation of the breast, said compression unit including:
a compression element permeable for X-rays and ultrasound and disposed to enable said ultrasound unit to be guided along said compression element over the breast;
a volume-variable and/or shape-variable positioning element for acting on the breast and configured to cause the breast to be deformed and to rest against said compression element;
said positioning element being a selectively fillable and emptiable cushion formed with at least one chamber, and wherein a volume-variability and/or a shape-variability of said positioning element is effected by filling said at least one chamber of said positioning element with a filling medium or by emptying said at least one chamber of said positioning element.

The advantages and embodiments explained below in connection with the X-ray examination device also apply analogously to the method according to the invention and vice versa.

In other words, the X-ray examination device according to the invention is embodied as a combined X-ray/ultrasound examination device and includes an X-ray tube and an X-ray detector for recording at least one X-ray image of a breast and an ultrasound unit for recording at least one ultrasound image of the breast and a compression unit for compression and fixation of the breast, wherein the compression unit comprises a compression element penetrable by X-rays and ultrasound and wherein the ultrasound unit can be guided over the breast along the compression element and is characterized by a volume-variable and/or shape-variable positioning element for acting on the breast such that the positioning element deforms the breast and the breast rests on the compression element, wherein the positioning element is embodied as a fillable and emptiable cushion comprising at least one chamber and wherein the volume-variability and/or shape-variability of the positioning element can be effected by filling at least one chamber of the positioning element with a filling medium and/or by emptying at least one chamber of the positioning element.

A method according to the invention for positioning a breast in an X-ray examination device embodied as a combined X-ray/ultrasound examination device with an X-ray tube and an X-ray detector for recording at least one X-ray image of a breast and with an ultrasound unit for recording at least one ultrasound image of the breast and with a compression unit for compression and fixation of the breast, wherein the compression unit comprises a compression element penetrable by X-rays and ultrasound and wherein the ultrasound unit can be guided over the breast along the compression element, is characterized in that the breast is positioned using a volume-variable and/or shape-variable positioning element for acting on the breast such that the positioning element deforms the breast and the breast rests on the compression element, wherein the positioning element is embodied as a fillable and emptiable cushion comprising at least one chamber and wherein the volume-variability and/or shape-variability of the positioning element is effected by filling at least one chamber of the positioning element with a filling medium and/or by emptying at least one chamber of the positioning element.

With the invention, pain on the compression or fixation of the breast during X-ray examinations is reduced or avoided altogether. In addition, in a combined X-ray/ultrasound examination, the invention enables improved coupling of the breast to the ultrasound transducer and hence improved coverage for the ultrasound recording with identical breast positioning.

A core concept of the invention is to use the positioning element to perform defined positioning of the breast for a medical imaging method, in particular 2D/3D mammography or tomosynthesis, including in combination with ultrasound imaging in that the positioning element is embodied as a fillable and re-emptiable cushion, wherein the filling and emptying is carried out in a targeted manner such that a contact surface of the cushion acts on a large area of the breast and in this way shapes and/or moves the breast. By virtue of this shaping and/or moving of the breast, the positioning element contributes to the desired positioning of the breast in the compression unit. In other words, the positioning element effects or supports positioning of the breast in a desired image recording position.

As a result, the breast in the compression unit is acted on from several sides, namely, on the one hand, by the compression elements of the compression unit, i.e., for example, the X-ray detector as the lower compression plate and a movable compression element, which can be embodied as an upper compression plate or a fabric, thus typically from below and from above, and, on the other hand, by the shape-variable and/or volume-variable positioning element, which rests on at least one side of the breast, for example on the lower side of the breast or the upper side of the breast, but preferably on several sides of the breast, in particular also laterally so that it is not only able to raise or lower the breast but also act on it laterally and thus cause it to adopt a desired shape. As a result, it is not only easier to bring to bring the breast into a desired position for the X-ray recording. It is also possible to achieve a particularly homogeneous or uniform, preferably all-round, force distribution on the breast, which results in improved fixation of the breast and also in reduced pain. Since, instead of hard surfaces, now at least partially, but preferably exclusively, flexible or yielding bodies, including the gauze, are used to act on the breast with the required contact force or the required pressure, the targeted compression or fixation is also perceived as particularly “soft” thus resulting in increased patient comfort. A suitable force or pressure distribution in accordance with the existing breast shape or anatomy in particular makes it possible to avoid unnecessarily high pressure on regions of the breast close to the breast wall. In the case of an evenly distributed application of force over the entire breast during compression, with the methods known from the prior art, these regions are exposed to particularly high pressures, which cause pain to the patients.

In addition, the positioning element enables advantageous positioning of the breast for ultrasound recording with a combined X-ray/ultrasound examination, in addition to or in conjunction with the compression and fixation of the breast already achieved by the compression unit. The positioning element enables the regions of the breast, which, during conventional compression and fixation by the compression unit, do not lie on the compression surface of the compression element, to rest thereupon so that these regions are also able to produce an ultrasound recording when the ultrasound unit is guided over the breast along the compression element. In other words, the positioning element is used as an additional positioning or fixation means for certain segments of the breast.

In other words, compression of the breast such as is effected in order to perform the X-ray recordings (mammography and/or tomosynthesis) can simultaneously enable the breast to be fixed in a manner advantageous for the performance of ultrasound recordings. Since, when examination device according to the invention is used, neither the location nor the compression of the breast changes when the imaging modalities are changed, both methods can be used with constant positioning, thus enabling image contents to be correlated with in a comparatively simple manner.

The use of the positioning element enables positioning of the breast advantageous for the ultrasonic coupling and the desired breast covering to be achieved in a simple manner. The positioning element that rests at least on segments of the breast enables the breast or a region of the breast to be raised, or to be more precise, lifted up by the X-ray detector and moved onto the underside of the compression element until it rests on the compression surface. Therefore, conventional devices only have to be supplemented by a suitable positioning element and a filling and emptying mechanism in order to implement the invention. Therefore, the invention can also be retrofitted onto existing devices.

Other features which are considered as characteristic for the invention are set forth in the appended claims.

Although the invention is illustrated and described herein as embodied in a X-ray examination device and a method of placing a breast for X-ray examination, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is a side view of a combined X-ray/ultrasound examination device according to the prior art;

FIG. 2 is a side view of a combined X-ray/ultrasound examination device according to the invention; and

FIG. 3 is a front view of the combined X-ray/ultrasound examination device according to the invention.

All the figures show the invention in schematic form only and with their essential components. Herein, the same reference numbers correspond to elements with the same or comparable function.

### DETAILED DESCRIPTION OF THE INVENTION

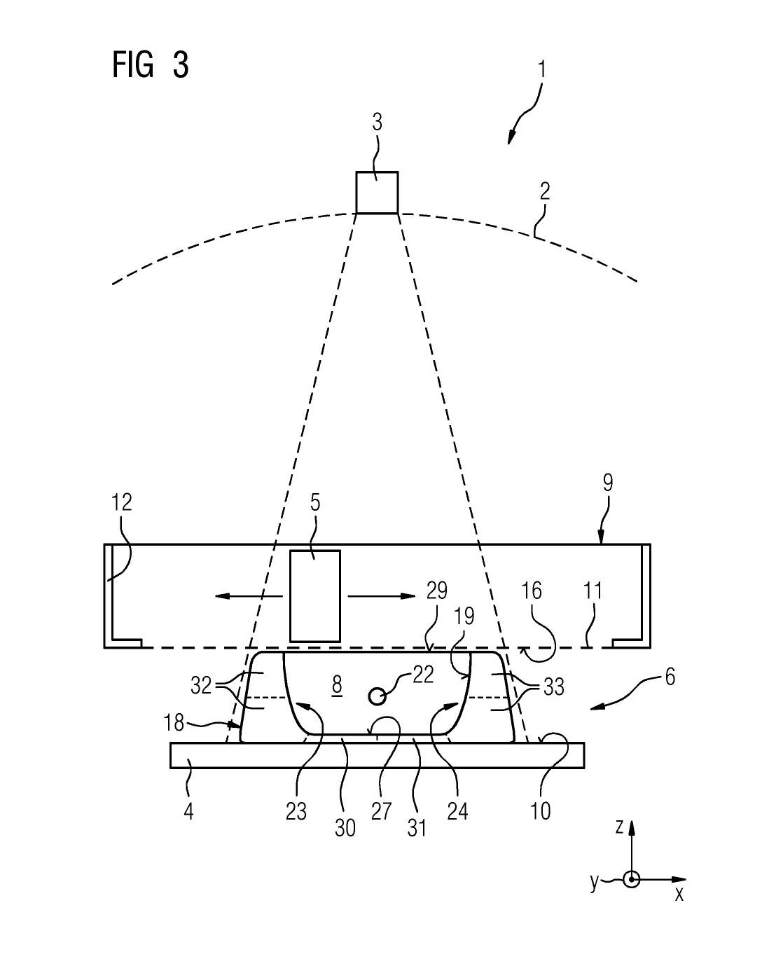

Referring now to the figures of the drawing in detail, there is shown a schematic representation of the parts of a combined X-ray/ultrasound examination device 1, which is suitable for performing mammography and tomosynthesis procedures. This examination device 1 comprises an X-ray tube 3 (cf. FIG. 3) that can be moved along a trajectory 2 with an associated X-ray detector 4 and an ultrasound unit with an ultrasound transducer 5 integrated in the compression unit, i.e. an ultrasound probe that transmits and receives the ultrasound waves, converts them into electrical signals and forwards them for further processing. Alternatively, the ultrasound unit can also comprise an ultrasound transmitter that interacts with a suitably placed ultrasound receiver (not shown).

The compression arrangement 6 includes an upper compression unit and a lower compression unit. The compression units can be positioned on opposite sides of an intermediate region of interest 7, wherein the X-ray tube 3 can be positioned such that its X-rays pass through the region of interest 7 and then strike the detector 4. An X-ray cone emitted by the X-ray tube 3 and propagated in the direction of the detector 4 is indicated in FIG. 3. During the X-ray recording, the ultrasound transducer 5 is positioned outside the X-ray cone.

In the present case, the detector 4, which can be embodied as a digital flat detector, serves as the lower compression unit. For a breast examination, a patient's breast 8 is positioned directly on the detector 4. The upper compression unit, here in the form of a compression trough 9, fixes and compresses the breast to be examined 8 between the compression trough 9 and the upper side 10 of the detector 4. The compression trough 9 is embodied toward the detector 4 with a flexible compression element in the form of a fabric 11, here gauze is used for this purpose, which is tensioned in a frame 12. This fabric 11 forms a compression surface 16 that is penetrable by X-rays and ultrasound and embodied to rest on the breast 8. Herein, the fabric 11 substantially spans the entire floor region of the compression trough 9.

It can in particular be provided that the fabric held in the compression trough 9 is a single-use article. This means that there is no need for time-consuming cleaning of the fabric after use to remove the contact gel. To this end, the frame 12 of the compression trough 9 can be embodied such that the fabric 11 is tensioned therein, in particular comprises suitable tensioning elements. However, the fabric 11 can also be glued to the frame 12 or attached detachably thereto in some other manner. Alternatively, it is also possible for the entire frame 12, together with the fabric 11, to be embodied as a single-use only component that is separable from the compression trough 9.

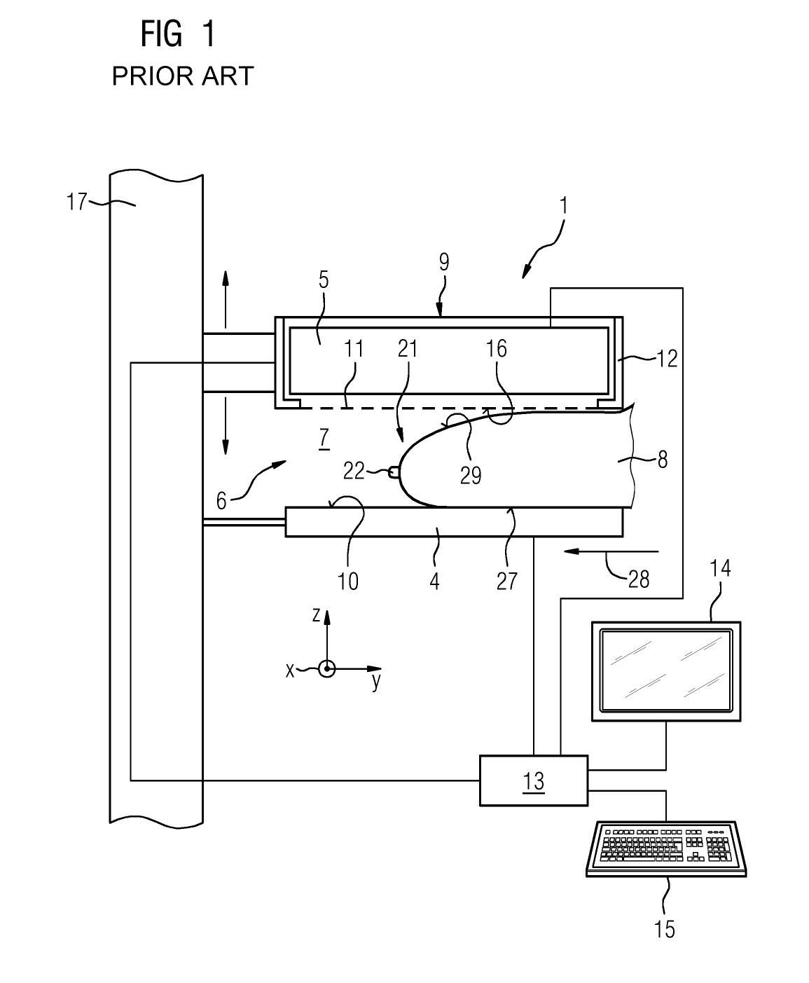

FIG. 1 depicts a control and computing unit 13 belonging to the examination device 1 with an associated screen 14 and input unit 15. This is used to control the examination device 1, i.e. both to control the X-ray tube 3 and the detector 4 and to control the ultrasound transducer 5, and is also used, for example, to perform image processing algorithms for the generation of 2D and/or 3D images. FIG. 1 shows that, with the methods used in the prior art, the anterior region 21 of the breast 8 in the region of the nipple 22 is not connected to the ultrasound transducer 5.

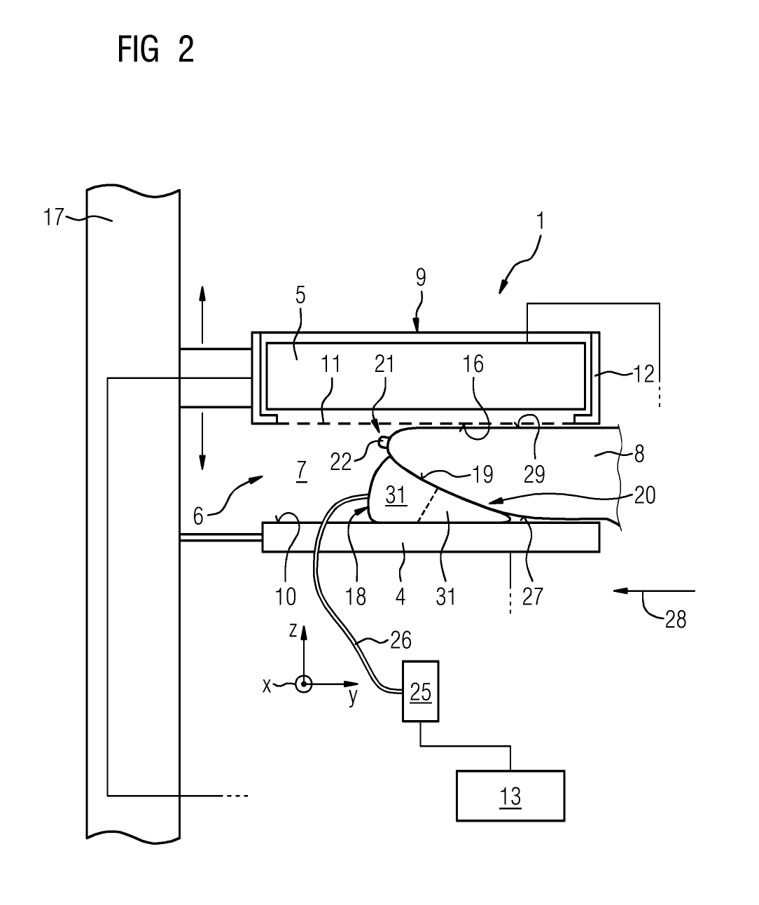

When the breast 8 has been compressed and fixed by the compression arrangement 6 and when additional positioning of the breast 8 has been performed with the aid of a positioning element 18, as explained in more detail below in connection with FIGS. 2 and 3, the X-ray tube 3 and the detector 4 are used to record first images of the breast 8. Then, while retaining compression and fixation of the breast 8, it is possible to record second images with the aid of the ultrasound transducer 5 in that the ultrasound transducer 5 is guided over the breast 8 along the compression surface 16. Compression of the breast 8 is maintained during image recording and between recording of the images, as are the position and the viewing angle of the respective recording.

The individual 2D X-ray images recorded with the X-ray detector 4 can be processed in the control and computing unit 13 by means of a reconstruction method to produce a 3D dataset. X-ray images can with be superimposed with ultrasound images. The different scan results are depicted and superimposed or fused by means of a suitable image processing procedure. This generates a visual depiction on the screen 14 or another reproduction device. Herein, it is, for example, possible for an X-ray image and an ultrasound image to be depicted superimposed or synchronously. It is then particularly easy for a physician to analyze the breast tissue examined using the X-ray and ultrasound images with an unchanged location.

Different X-ray recordings (mammography, tomosynthesis) can be performed alone or in combination with ultrasound recordings. An ultrasound recording can also be performed independently of an X-ray recording.

FIG. 1 and FIG. 2 show similar side views. FIG. 2 shows a schematic side view of an examination device 1 according to the invention which, unlike the case of FIG. 1, is equipped with a positioning element 18 according to the invention. The detector 4 and the compression trough 9 are arranged on a vertical support element 17 of the examination device 1, here a stand. The X-ray tube 3 can also be attached to the support element 17 or even to a separate holding arm (not shown) of the examination device 1. The compression units (detector 4 and compression trough 9) are mounted on the support element 17 such that the compression trough 9 can be lowered to compress a breast 8 located in the region of interest 7 (indicated by arrows). The detector 4 is preferably used as a static support but can also be mounted in a vertically adjustable manner.

In the compression trough 9, the ultrasound transducer 5 is placed resting on the compression surface 16 in a horizontally and vertically movable manner, i.e. guided over the fabric 11 lying on the breast 8. This ultrasound transducer 5 can be moved horizontally by means of a guiding and moving unit (not shown) in the X- and/or Y-direction in the plane defined by the compression trough 9 (indicated by arrows in FIG. 3). A motor drive which is connected to the guiding and moving unit and provided to move the ultrasound transducer 5 integrated in the compression trough 9 is also provided, but not shown.

For better coupling of the ultrasound waves to the breast 8, an ultrasound coupling gel (contact gel) can be applied to the respective compression element, here the fabric 11, by means of a dispensing unit (not shown).

Prior to placing the breast 8 on the X-ray detector 4, an inflatable air cushion 18 serving as a positioning element 18 is arranged there, see FIG. 2. The air cushion 18 can be divided into individual chambers or sections. The air cushion 18 preferably comprises a plurality of chambers 30, 31, . . . which are separate from one another or can be separated from one another, which can optionally be filled with air or emptied of air as a result of which the shape and/or volume of the chambers 30, 31, . . . , and hence the shape and/or volume of the air cushion 18, can be changed. To this end, the chambers 30, 31, . . . can preferably be filled and emptied independently of one another. In other words, the pressure inside the chambers 30, 31, . . . can be changed individually for each chamber and in a targeted manner. This provides the air cushion 18 with volume-variability and/or shape-variability. In other words, the shape and/or volume of the air cushion 18 changes in dependence on its degree of filling. For reasons of clarity, neither FIG. 2 nor FIG. 3 depicts all the chambers 30, 31, . . . of the air cushion 18.

Instead of air, it is also possible to use another suitable gas mixture to fill the air cushion 18. In general, depending upon the application and embodiment of the system, instead of a pneumatic filling system, it is also possible to use a hydraulic filling system and the filling medium can be any fluid suitable for this purpose, wherein the advantages and properties explained below in connection with an air cushion 18 can be correspondingly transferred to other embodiments.

The air cushion 18 provides a plurality of contact surfaces 19 for the breast 8 on its outer surfaces. These are used before, during and/or following the lowering of the upper compression unit 9 for acting on the breast 8 in a planar manner in order to position the breast in the compression arrangement 6, i.e. between the two compression units 4, 9, and/or in order to compress and/or fix the breast. In other words, the contact surfaces 19 of the air cushion 18 act on the breast 8 in that their filling causes them to rest on the breast 8 and hence fix the breast 8 or segments or parts of the breast 8 in a specific position and/or in that, in the further course of the positioning process, in particular during or as a result of further filling of the air cushion 18 and an increase in the internal pressure in the chamber, the breast 8 or segments or parts of the breast 8 change shape and/or move into a specific target position. The time of the filling of the air cushion 18 or the time regime for filling and/emptying the individual chambers 30, 31, . . . of the air cushion 18 is defined in dependence on the breast 8 to be positioned, in particular the size and shape of the breast 8, and the target positioning to be achieved and in coordination with the compression process by the lowering of the compression unit 9.

The air cushion 18 is inter alia used, in particular with its chambers 30, 31, . . . arranged below the breast 8, to raise segments of the breast 8 lying thereupon such that the raised segment 21 of the breast 8 rests on the compression surface 16 arranged above the breast 8, i.e. here the gauze 11, from below. However, the breast 8 can also be completely, i.e. not only in segments, raised by the air cushion 18.

Simultaneously, the air cushion 18, in particular its chambers 32, 33, . . . arranged to the side of the breast 8, is used to shape the breast 8, preferably such that the breast 8 is preferably moved or compressed in a wedge or cone-shape in the direction of the compression surface 16, and, to be precise, in particular such that the breast surface lying on the compression surface 16 is larger than the breast surface lying on the X-ray detector 4 or pointing in the direction of the X-ray detector 4. It is also possible for the chest wall regions, in particular the lateral breast wall regions and the anterior breast wall region 21 around the nipple 22 to be shaped and formed by suitable filling of the chambers 30, 31, . . . of the air cushion 18 such that its location or course are optimal for the desired image recordings.

Unlike the case with conventional compression known from the prior art with the aid of rigid compression plates, with the application of the positioning element 18 according to the invention, the breast shape of the compressed breast 8 changes. In other words, the breast 8 compressed according to the invention has a different shape, in particular another cross-sectional shape than with conventional methods that position the breast without the positioning element 18. In addition, in certain circumstances, there is also a change in the curvature of the gauze 11 on which the breast 8 rests.

The number and arrangement of the chambers 30, 31, . . . can vary from air cushion to air cushion in dependence on the respective application. Typically, lower chambers 30, 31, . . . are provided which can be placed directly on the X-ray detector 4 and on which the breast 8 is arranged (rests). Additionally, preferably lateral chambers 32, 33, . . . are provided that are arranged to the sides of the placed breast 8 such that, viewed in the longitudinal direction of the breast 28, a substantially U-shaped cross section of the air cushion 18 results, see FIG. 3. Further additionally, preferably anterior chambers are provided (not shown) that are located in front of the nipple 22. These anterior chambers enable the breast 8 to be enclosed on three sides.

Preferably a plurality of chambers 30, 31, . . . are arranged in the longitudinal direction of the breast 28 next to one another so that different segments of the breast 8 in the longitudinal direction of the breast 28 can be acted upon in different ways, i.e. for example a posterior segment, close to the pectoral muscle, a middle segment 20 and an anterior segment 21, close to the nipple, of the breast 8. In addition, preferably a plurality of chambers 30, 31, . . . are arranged next to one another transverse to the longitudinal direction of the breast 28 so that different segments of the breast 8 transverse to the longitudinal direction of the breast 28 can be acted on differently, i.e. for example a right lateral segment 23 of the breast 8, when viewed in the direction of the nipple 22, or a left lateral segment 24 of the breast 8, when viewed in the direction of the nipple 22.

In other words, individual inflation of the air cushion 18 makes it possible exert a different pressure, viewed from the right and left or in the transverse direction, i.e. transverse to the longitudinal direction of the breast 28, on the breast 8 so that, for example, the right side 23 the breast 8, when viewed in the longitudinal direction of the breast 28, is acted on more strongly than left side 24 the breast 8, when viewed in the longitudinal direction of the breast 28 as a result of which, for example, the right side 23 the breast 8 is raised to a greater degree or pressed to a greater degree on the compression surface 16 than the left side 24 of the breast 8 or vice versa. This makes it possible to set the raising of the breast 8 or the positioning thereof in a general manner and the desired end position of the breast 8 on the compression surface 16 individually for each breast.

In particular, the lateral chambers 32, 33, . . . and the anterior chambers can be arranged, not only next to one another, but also one above the other. This, for example, enables a segment of the breast 8 close to the underside of the breast 27 to be acted upon differently than a segment of the breast 8 close to the upper side of the breast 29. In completely filled state, the chambers 30, 31, . . . can achieve a height corresponding at least to the distance between the X-ray detector 4 and the compression unit 9 in completely compressed final state. In other words, when in its final recording position, the breast 8 can be completely surrounded laterally by chambers 30, 31, . . . of the air cushion 18 and acted upon by the contact surfaces 19 thereof.

In the simplest case, the air cushion 18 is the same thickness all over or the air cushion 18 is equipped with identical chambers 30, 31, . . . over its entire length. However, advantageously, depending upon the region of the air cushion 18 in which the chambers 30, 31, . . . are located, chambers 30, 31, . . . of different sizes or different shapes can be provided. For example, in the inflated, filled state, the chambers 30, 31, . . . of the air cushion 18 arranged in the region of the anterior side of the breast, i.e. in the anterior region 21 of the breast 8 to be raised, can be thicker (higher) below the breast 8 than the chambers located in a rear region below the breast 8 in which the breast 8 is already compressed and fixed between the compression unit 9 and the X-ray detector 4.

The filling and emptying of the chambers 30, 31, . . . always takes place in a defined, i.e. non-arbitrary manner. In particular, the sequence and/or the speed of the filling and emptying of the chambers 30, 31, . . . can be coordinated with the process of the compression and fixation of the breast 8 by means of the compression arrangement 6 and adapted to the respective application. For example, during the positioning the breast 8, it is not necessary for all the chambers 30, 31, . . . of the air cushion 18 to be, or become, filled. It is also possible that, during the positioning process, one chamber 30, 31, . . . is first filled, and then, for example at the same time as the filling of an adjacent chamber, emptied again, for example in order to achieve a specific shape of the breast 8. It is also possible for a chamber 30, 31, . . . to be filled and emptied several times during positioning or for the filling (or emptying) to take place in several temporally separate steps. At the start of the positioning process, chambers 30, 31, . . . can be already partially or completely filled in order, for example, to provide a preferred starting position of the breast 8 for the subsequent positioning process.

During the filling, the first regions of the air cushion 18 to be filled are those offering little resistance to the filling process. These are typically regions in which the chambers 30, 31, . . . can be filled without having to lie against the breast 8. In this way, first all the chambers 30, 31, . . . that do not directly contribute to the action on the breast are filled. When the air pressure is further increased, the breast 8 is acted on as desired in the selected regions.

The filling rate can be regulated in dependence on the degree of filling the respective chamber 30, 31, . . . , in particular such that the filling rate drops as the chamber 30, 31, . . . approaches its maximum degree of filling or its conventional degree of filling and/or in dependence on the distance set between the X-ray detector 4 and the compression unit 9. This enables the time required for the filling to be minimized. To empty the chambers 30, 31, . . . , the filling air can be simply let out of the chambers 30, 31, . . . , for example through a discharge valve or this like. In the case of time-critical applications, the chambers 30, 31, . . . can also be emptied more quickly by means of pumps.

In the simplest case, the air cushion 18 is not inflated at the start of the positioning process. The compression unit 9 is then lowered until the gauze 11 presses gently on the upper side 29 of the breast 8 and prefixates the breast. The air cushion 18 is now filled in accordance with the desired target position of the breast 8; the breast 8 is enclosed by the air cushion 18 and pressed against the gauze 11.

The chambers 30, 31, . . . of the air cushion 18 are filled and emptied by means of a pneumatic system to be connected to the air cushion 18, to be more precise the number of chambers 30, 31, . . . , as indicated in FIG. 2, with at least one preferably (electric) motor operated pump 25 and further elements, such as, for example, a number of valves, manifolds, connecting lines 26 etc. If the chambers 30, 31, . . . provided can be filled singly or individually, they have a locking function thus enabling the filling of selected chambers 30, 31, . . . to be suppressed or released as desired. The design and function of such a system are known to the person skilled in the art and therefore do not require further explanation at this point. Herein, all elements produced from materials that are not penetrable by X-rays or generally interfere with image recording are preferably arranged outside the region of interest in order to avoid image artifacts and the like during imaging.

In the simplest case, the pneumatic system can be operated manually. For example, the pump 25 and the operating elements, such as shut-off or passage elements for the connecting lines 26, can be actuated by hand.

In another variant, the operator is assisted by a visual display showing the location and shape of the breast 8. As before, the operating elements are operated manually. The display depicts the current breast positioning, preferably by means of an image of the breast 8 from above (top view) and/or from the front. This display device is, for example, the screen 14.

In a further embodiment, remote-controlled operation of the operating elements of the pneumatic system is provided. In this case, once again, a visual display of the breast position is provided. Herein, it is again possible to use the screen 14 as a display device together with a separate input unit 15. Alternatively, the display device simultaneously serves as an input unit for manual control, to which end it is possible to use a touchscreen or the like as a user interface (not shown).

In an alternative embodiment, semi-automatic or fully automatic operation of the operating elements using ascertained data is provided. While, in semi-automatic mode, the visual display is used to perform or confirm individual operator actions by means of an input function, in fully automatic mode, no interaction with the operator is provided, apart from an optional confirmation or release function.

It is then provided that, in dependence on the size and location of the breast 8, the breast positioning can be performed automatically by means of automatic filling and emptying in coordination with the conventional compression and fixation procedure by means of the compression arrangement 6, here the lowering of the compression unit 9.

The ascertained data is preferably breast-related data, in particular data relating to the size, location and/or position of the breast 8. In addition, the ascertained data is preferably data relating to the air cushion, in particular data on the filling status of the chambers 30, 31, . . . or chamber pressure. The filling and/emptying of the chambers 30, 31, . . . or a change to the chamber pressure is performed automatically with the aid of a control unit for controlling the filling/emptying mechanism, wherein this control unit processes control commands created using the ascertained data. This control unit is preferably the control and computing unit 13 of the examination device 1, which also implements this function. This takes place using a suitable algorithm that calculates optimum compression and/or fixation and/or positioning of the breast 8 based on the ascertained data and the type of recording set (for example mammography, tomosynthesis, combined recording) and, optionally after confirmation by the operator, implements, controls and, if necessary, corrects them.

The pressure inside the chambers 30, 31, . . . can be acquired by means of pressure sensors (not shown), wherein, to avoid artifacts, these are preferably arranged outside the air cushion 18. The length, width and height or the location and/or position of the breast 8 can be acquired with the aid of location- and/or distance sensors (not shown) or with the aid of a photographic or video camera (not shown) placed in the vicinity of the examination device, wherein the photographic or video data or all the acquired data is conditioned or further processed in order to be further used in the control and computing unit 13. In addition, it is also possible for other suitable parameters to be acquired by the sensor system and used via the control unit 13 to control the pneumatic system, in particular to control the pump 25 and the operating elements, i.e., for example, the locking elements.

In order not to influence the imaging, in particular to avoid the occurrence of stray radiation and image artifacts, the air cushion 18 preferably made of a material penetrable by X-rays, in particular a flexible plastic material, and the chamber walls have a comparatively low material thickness. The air cushion 18 or the chambers 30, 31, . . . thereof are also flexible to a certain degree in filled state, so that they are able to adapt to the shape of the breast 8 to be acted upon with the desired pressure. This achieves particularly gentle handling of the breast 8 during positioning, in particular shaping and/or movement.

In addition to different filling of individual chambers 30, 31, . . . of the air cushion 18, i.e. filling with different pressures, or even instead of filling with different pressures, in one embodiment of the invention, the establishment of the shape and volume of the air cushion 18 can be influenced in a targeted manner such that different material thicknesses of the chamber walls are used, at least for specific segments or parts of the air cushion 18 or the chambers 30, 31, . . . thereof. In other words, first regions, which, with the same filling pressure, are to have a lower volume than second regions are equipped with thicker chamber walls or second regions, which are to have a larger volume, are provided with thinner chamber walls. As a result, it is possible to achieve a specific desired shape of a chamber 30, 31, . . . with the same internal pressure without subdivision into regions with different pressure. This enables the number of the chambers 30, 31, . . . required, and hence the number of pneumatic connections required etc., to be reduced.

Preferably, the air cushion 18 is embodied for one-time use, i.e. it does not have to be cleaned after the recordings. In the exemplary embodiment depicted, the air cushion 18 is attached to the X-ray detector 4. To enable the air cushion 18 to be exchanged quickly, it is preferably provided with quick-disconnect connectors to form a latching, snap-in or clamping connection or a Velcro fastener (not depicted) so that is possible to release and attach the air cushion 18 in a few simple steps. Preferably, the air cushion 18 has an antibacterial coating on its outer side or is endowed with antibacterial properties in some other way.

If the air cushion 18 substantially completely covers the bearing surface of the X-ray detector 4 that can be occupied by the breast 8, this prevents wetting, and hence contamination, of the X-ray detector 4 by ultrasound gel. Therefore, there is no need to clean the X-ray detector 4. Simultaneously, the use of an air cushion 18 that substantially completely covers the X-ray detector 4 ensures that, regardless of their size, breasts 8 can be positioned by the air cushion 18 after being placed on the X-ray detector 4.

Some of the chambers 30, 31, . . . of the air cushion 18 are preferably arranged such that, in filled state, they are guided upward from the underside 27 of the breast 8 in the region of the nipple 22. This ensures that the regions of the breast 8 close to the breast wall, in particular in the region of the nipple 22, are raised by the positioning element 18, i.e. the segments of the breast 8 with a tissue thickness that is so low that, following conventional compression and fixation by means of the compression unit 9, they do not rest on the compression surface 16 and are therefore unable to contribute to an ultrasound image. Typically, in other words, the anterior side of the breast or the anterior breast region 21 is raised.

Following compression and fixation of the breast 8 by means of the compression unit 9 and following the above-described positioning of the breast 8, the required X-ray images of the breast 8 are recorded. This is followed by the ultrasound recordings. This achieves particularly good coupling of the breast 8 to the ultrasound transducer 5 and hence particularly good coverage of the breast surface of up to 100 percent. The compression and fixation of the breast 8 during the recording of the X-ray images, including the additional positioning by the air cushion 18, is maintained during the recording of the ultrasound images. The constant positioning of the breast 8 during the entire examination period makes it easier to correlate the image contents of the two modalities thus simplifying local assignment of suspect regions. This results in improved diagnostics.

The use of the invention enables patient-specific and targeted breast positioning to be performed in a simple manner even without using compression units 9 of different sizes. Breast positioning that is optimized on a case-by-case basis enables account to be taken individually of special clinical questions and typical breast shapes. Herein, it is not only possible to connect compression units 9 according to the invention, which are not described in any more detail, to the examination device. It is also possible to use conventional compression units 9 with flat, rigid compression surfaces, for example made of plexiglass, in some cases, for example when only an X-ray examination (mammography and/or tomosynthesis) of the breast 8 is to be performed, but not an ultrasound examination. In this case, the positioning element, here the air cushion 18, can be used exclusively or additionally as a compression unit 9 and press the breast 8 onto the X-ray detector 4. In this case, i.e. when exclusively X-ray recordings are to be made, it is also possible for the air cushion 18 to be embodied such that chambers are provided above the breast 8. These upper chambers (not depicted) can be used to form a compression surface 16 that is individually shaped for each breast and shape the breast 8 from above. These upper chambers can be used alone or in combination with lower and/or lateral chambers 30, 31, . . . .

In all cases described, instead of one single air cushion 18 it is possible to use a plurality of air cushions 18. These are then preferably connected mechanically to one another to produce a group of positioning elements that can be operated similarly to the individual chambers 30, 31, . . . of a single positioning element.

Although the invention was illustrated and described in more detail by the preferred exemplary embodiment, the invention is restricted to the disclosed examples and other variations can be derived herefrom by the person skilled in the art without departing from the scope of protection of the invention. In particular, the statements made in connection with the use of compression troughs are in all cases also applicable to other types of compression unit.

The following is a summary list of reference numerals and the corresponding structure used in the above description of the invention:
1 Examination device
2 Trajectory
3 X-ray tube
4 X-ray detector, detector
5 Ultrasound unit, ultrasound transducer
6 Compression arrangement
7 Region of interest
8 Object, breast
9 Upper compression unit, compression trough
10 Lower compression unit, detector top side
11 Compression element, fabric, gauze
12 Frame
13 Control and computing unit
14 Screen
15 Input unit
16 Compression surface
17 Support element, stand
18 Positioning element
19 Contact surface
20 Middle breast region
21 Anterior breast region
22 Nipple
23 Right lateral segment
24 Left lateral segment
25 Pump
26 Connecting line
27 Underside of breast
28 Longitudinal direction of breast
29 Top side of breast
30 Lower chamber (right)
31 Lower chamber (left)
32 Lateral chamber (right)
33 Lateral chamber (left)

1. An X-ray examination device being a combined X-ray and ultrasound examination device, the X-ray examination device comprising:
an X-ray tube and an X-ray detector for recording an X-ray image of a breast;
an ultrasound unit for recording an ultrasound image of the breast; and
a compression unit for compression and fixation of the breast, said compression unit including:
a compression element permeable for X-rays and ultrasound and disposed to enable said ultrasound unit to be guided along said compression element over the breast;
a volume-variable and/or shape-variable positioning element for acting on the breast and configured to cause the breast to be deformed and to rest against said compression element;
said positioning element being a selectively fillable and emptiable cushion formed with at least one chamber, and wherein a volume-variability and/or a shape-variability of said positioning element is effected by filling said at least one chamber of said positioning element with a filling medium or by emptying said at least one chamber of said positioning element.

2. The X-ray examination device according to claim 1, wherein said positioning element is an inflatable air cushion.

3. The X-ray examination device according to claim 1, wherein the volume-variability and/or shape-variability of said positioning element is influenced by a material thickness of the walls of said at least one chamber.

4. The X-ray examination device according to claim 1, wherein said at least one chamber of said positioning element is one of a plurality of chambers that can be selectively filled and emptied independently of one another.

5. A method for positioning a breast in an X-ray examination device, the X-ray examination device being a combined X-ray/ultrasound examination device with an X-ray tube and an X-ray detector for recording at least one X-ray image of the breast, with an ultrasound unit for recording at least one ultrasound image of the breast, and with a compression unit for compression and fixation of the breast, wherein the compression unit includes a compression element penetrable by X-rays and ultrasound and wherein the ultrasound unit can be guided along the compression element over the breast, the method comprising:
providing a volume-variable and/or shape-variable positioning element being a selectively fillable and emptiable cushion formed with at least one chamber;
positioning the breast by using the positioning element to act on the breast and to deform the breast such that the breast rests against the compression element; and
effecting a volume-variability and/or shape-variability of the positioning element by filling the at least one chamber of the positioning element with a filling medium and/or by emptying the at least one chamber of the positioning element.

6. The method according to claim 5, which comprises acting on the breast with the positioning element before, during and/or after a compression and fixation of the breast, such that the breast rests on the compression element before the at least one X-ray image of the breast is recorded.

7. The method according to claim 6, which comprises maintaining the compression and fixation of the breast provided during the recording of the at least one X-ray image during the recording of the at least one ultrasound image.

8. The method according to claim 5, which comprises using the positioning element as the compression unit.

9. The method according to claim 5, which comprises selectively filling or emptying the chambers of the positioning element in a defined sequence.

10. The method according to claim 5, which comprises altering a pressure inside the chambers in a defined manner.

11. The method according to claim 5, which comprises acquiring breast-related and/or positioning-element-related data and filling or emptying the chambers in accordance with the data.

12. The method according to claim 5, which comprises displaying a position of the breast on a display device, and supporting an operator with the display in an actuation of operating elements of a pneumatic system for filling and emptying the chambers of the positioning element.

13. The method according to claim 5, which comprises acquiring breast-related data and acquiring data on a filling status of the chambers or on a chamber pressure, and automatically filling or emptying the chambers of the positioning element using the acquired data.

14. The method according to claim 13, wherein the breast-related data are selected from the group consisting of a size, a location and a position of the breast.
